(19) United States
(12) Patent Application Publication
(10) Pub. No.: US 2009/0306587 A1
(43) Pub. Date: Dec. 10, 2009

(54) Sleeve Steering and Reinforcement

(76) Inventors: Zoran Milijasevic, Bayview (AU); Norman Booth, Wattle Grove (AU)

Correspondence Address: MORRISON & FOERSTER LLP, 755 PAGE MILL RD, PALO ALTO, CA, 94304-1018, US

(21) Appl. No.: 11/922,553

(22) PCT Filed: Jun. 20, 2006
(86) PCT No.: PCT/AU2006/000862

§ 371 Date: Mar. 12, 2008

Related U.S. Application Data
(60) Provisional application No. 60/692,848, filed on Jun. 20, 2005.

(51) Int. Cl. A61M 25/092; A61M 25/00

(52) U.S. Cl. 604/95.04; 604/526

(57) ABSTRACT

A sleeve steering and reinforcing device (10) includes a first, elongate, helical element (12) having spaced turns (14). At least one further, elongate, helical element (16) is co-axially arranged with the first helical element (12). The at least one further helical element (16) has a plurality of spaced turns (18) wound in an opposite direction to the turns (14) of the first helical element (12) such that the turns (14, 18) of the elements (12, 16) coincide at predetermined zones (20, 22), the zones (20, 22) being arranged along lines extending parallel to a longitudinal axis of the elements.

### CROSS-REFERENCE TO RELATED APPLICATIONS

The present application claims priority from United States of America Provisional Patent Application No. 60/692,848 filed on 20 Jun. 2005, the contents of which are incorporated herein by reference.

### FIELD OF THE INVENTION

This invention relates to sleeve steering and reinforcement More particularly, the invention relates to a sleeve steering and reinforcing device and to a sleeve including such a device.

### BACKGROUND TO THE INVENTION

In the use of catheters, a catheter is inserted into a patient's body via an introducer. The introducer or catheter is inserted into the vascular system of the patient's body and is steered to the desired site of the patient's body. At the site, a distal part of the catheter is urged out of the introducer to enable the required therapeutic action, be it diagnosis or treatment, to be taken by the clinician using the catheter.

To enable the clinician to steer the introducer to the desired site, at least the distal end of the introducer and/or the catheter (referred to below collectively as an “elongate device”) needs to be steerable. Conventionally, this has been achieved by inserting a shim such as a flat, metal strip, into the distal end of the elongate device to be steered. Pull wires are attached to a proximal end of the shim. The shim provides stiffness against bending in the plane of the shim and allows bending in a plane. transverse to the plane of the shim.

A problem with this arrangement is that there is very little stiffness in torsion provided by the shim. It will be appreciated that, because bending only in a single plane is possible with the shim, it is necessary for a clinician physically to rotate the elongate device about its longitudinal axis in order to achieve changes in direction of the distal end of the elongate device. Thus, without stiffness in torsion, there is not a 1:1 correspondence between a clinician rotating the proximal end of the elongate device and the resultant rotation at the distal end of the elongate device.

### SUMMARY OF THE INVENTION

According to the invention, there is provided a sleeve steering and reinforcing device which includes:

a first, elongate, helical element having spaced turns wound in a first direction; and

at least one further, elongate, helical element co-axially arranged with the first helical element, the at least one further helical element having a plurality of spaced turns wound in an opposite direction to the turns of the first helical element such that the turns of the elements coincide at predetermined zones, the zones being arranged along lines extending parallel to a longitudinal axis of the elements.

In this specification, the term “sleeve” is to be understood to mean any elongate tubular element whether having an open end or a closed end. In addition, the term “plane of stiffness” is to be understood as a plane in which there is resistance to bending of the device.

The device may comprise at least two helical elements. The elements may be formed by working a tubular work piece of appropriate material. For example, the work piece may be of stainless steel, nitinol, titanium, or other suitable biocompatible, resiliently flexible metal. Instead, the work piece may be of a suitable synthetic plastics material such as an appropriate polymer, for example, nylon. The work piece may be worked by removing material to define the turns of the helical elements.

Thus, the coinciding zones of the elements may be in the form of zones of intersection of turns of the elements.

It will, however, be appreciated that, instead, two helical spring-like structures of the appropriate pitch may be co-axially arranged to provide the coinciding zones.

The lines may lie out of a plane of stiffness, the plane of stiffness passing through a longitudinal axis of the elements.

In one embodiment, the pitch of the turns of one of the helical elements may be the same as the pitch of the turns of the other helical element. In that case, the coinciding zones of the turns may be arranged along lines which are spaced 180° from each other, the lines lying in a plane of bending in which bending of the device is facilitated. The plane of bending may pass through the longitudinal axis of the elements and may lie orthogonally relative to the plane of stiffness.

In another embodiment, the device may comprise two helical elements of oppositely directed turns with the pitch of the turns of one of the helical elements being different from the pitch of the turns of the other helical element The pitch of the turns of one of the helical elements may be twice that of the pitch of the turns of the other helical element. In this arrangement, three lines of coinciding zones of the turns may be formed with adjacent lines being spaced 120° apart

The device may include a control arrangement. The control arrangement may control bending of a distal end of the device. The control arrangement may comprise a plurality of elongate control members, such as control wires. In the case where the turns of the helical elements are of the same pitch, two control wires may be provided with the two wires lying in a plane transverse, more particularly, orthogonal, to the plane of stiffness, i.e. lying in the plane of bending. In the case where the pitch of the turns of one of the helical elements is twice that of the turns of the other helical element, three control wires may be provided with adjacent control wires being spaced 120° apart. In the latter case, the control wires may be in register with the lines of coinciding zones.

A stiffening collar may be arranged at least at coinciding distal ends of the elements. A distal end of each control wire may be secured at or adjacent the collar.

At least one further collar may be arranged proximally of the distal collar. A set of control wires may be associated with the at least one further collar as well as with the distal collar to provide a plurality of independently controllable sections of the device.

As indicated above, the helical elements may be of metal. Instead, or in addition, one or both helical elements may be of a polymeric material. Properties of the polymeric material of at least one of the helical elements may differ along the length of the at least one helical element to vary characteristics, for example, stiffness, of the at least one helical element along its length.

Further, a pitch of the turns of at least one of the helical elements may vary along the length of the at least one helical element to provide a variable radius of curvature of bending along the length of the device.

Still further, a pitch of the turns of at least one of the helical elements may vary along the length of the at least one helical element and the zones at which the turns of the helical elements intersect may be arranged along spirals to facilitate a twisting or snaking motion of the device.

The invention extends to a sleeve which includes an elongate tubular member; and

a sleeve steering and reinforcing device, as described above, carried by the tubular member.

### BRIEF DESCRIPTION OF THE DRAWINGS

FIG. 1 shows a three dimensional view of a sleeve steering and reinforcing device, in accordance with a first embodiment of the invention;

FIG. 2 shows a three dimensional view of a sleeve steering and reinforcing device, in accordance with a second embodiment of the invention;

FIG. 3 shows a side view of the device of FIG. 2;

FIG. 4 shows an end view of the device of FIG. 2; and

FIG. 5 shows a three dimensional view of a further embodiment of a sleeve steering and reinforcing device.

### DETAILED DESCRIPTION OF EXEMPLARY EMBODIMENTS

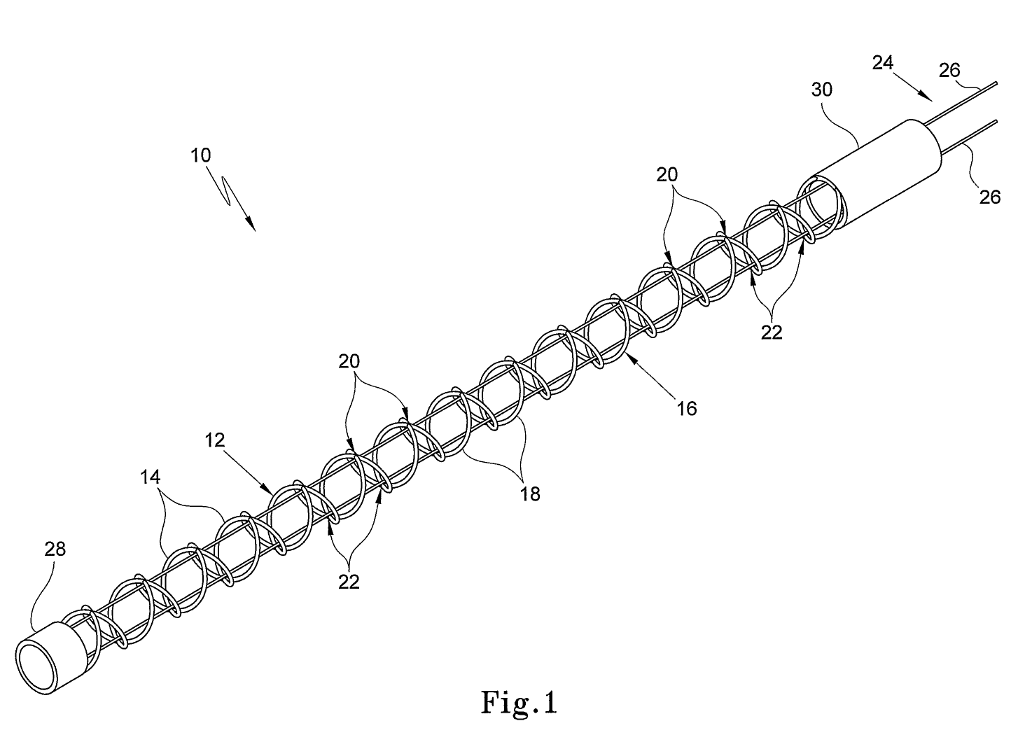

In FIG. 1 of the drawings, reference numeral 10 generally designates a sleeve steering and reinforcing device, in accordance with a first embodiment of the invention. The device 10 includes a first, elongate, helical element 12 having spaced turns 14. The device 10 further includes a second, elongate, helical element 16, once again, having spaced turns 18. The first element 12 and the second element 16 are co-axially arranged in register with each other. The turns 14 of the first helical element 12 are oppositely directed with respect to the turns 18 of the second helical element 16 to provide coinciding zones of intersection 20 and 22.

In the embodiment shown in FIG. 1 of the drawings, the pitch of the turns 14 of the helical element 12 is the same as the pitch of the turns 18 of the helical element 16.

The turns 14, 18 of the helical element 12, 16, respectively, coincide at the zones 20 and 22. The zones 20 are arranged along a longitudinally extending, imaginary line. Similarly, the zones 22 are arranged along a longitudinally extending, imaginary line extending parallel to the line on which the zones 20 are arranged. It will be appreciated that, because of the equal pitch of the turns 14 and 18, the lines are separated from each other by 180°. These imaginary lines effectively define regions of reduced stiffness of the device 10 and facilitate bending of the device 10 in a plane of bending in which the lines lie. The plane of bending is orthogonally arranged relative to a plane of stiffness, as defined. The benefit of the oppositely directed turns 14, 18 of the helical elements 12 and 16 is that torsional transmission from a proximal end of a sleeve (not shown), in which the device 10 is carried, to a distal end of the sleeve is facilitated.

The device 10 includes a control arrangement 24 for controlling steering of the sleeve in which the device 10 is arranged. In this regard, it is to be noted that the device 10 is, typically, carried at a distal end of a sleeve to be introduced into a patient's vascular system for steering a catheter assembly to a site in the patient's body to be treated. An example of the type of sleeve with which the device 10 is used is described in the Applicant's co-pending International Patent Application No. PCT/AU2005/00058 dated 20 Jan. 2005 and entitled “A catheter assembly with an adjustable loop”. The contents of that International Application are incorporated herein by reference. In that International Application, a catheter assembly is taught having a pair of nested sleeves. To facilitate steering of the distal end of each of the sleeves, a device 10 of the type described in this specification can be associated with, for example, be embedded in, a wall of the sleeve at a distal end of the sleeve.

The control arrangement 24 of the device 10 facilitates steering of the sleeve by means of a pair of control wires 26.

The device 10 includes a distal collar 28 and a proximal collar 30 between which the helical elements 12, 16 are arranged. The control wires 26 of the control arrangement are secured to an inner surface of the distal collar 28.

In this embodiment, the control wires 26 are secured to the collar 28 and are spaced 180° from each other to lie in the plane of bending. It will therefore be appreciated that, with this arrangement, the control wires 26 can be used for steering the device 10, and, accordingly, a sleeve in which the device 10 is carried, in the plane of bending orthogonal to the plane of stiffness.

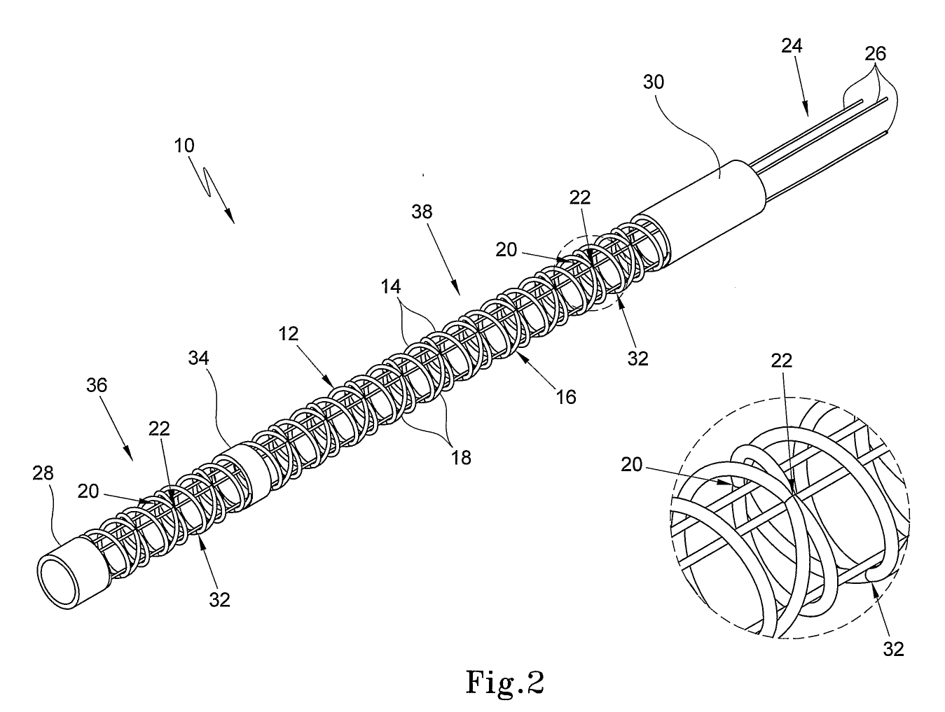

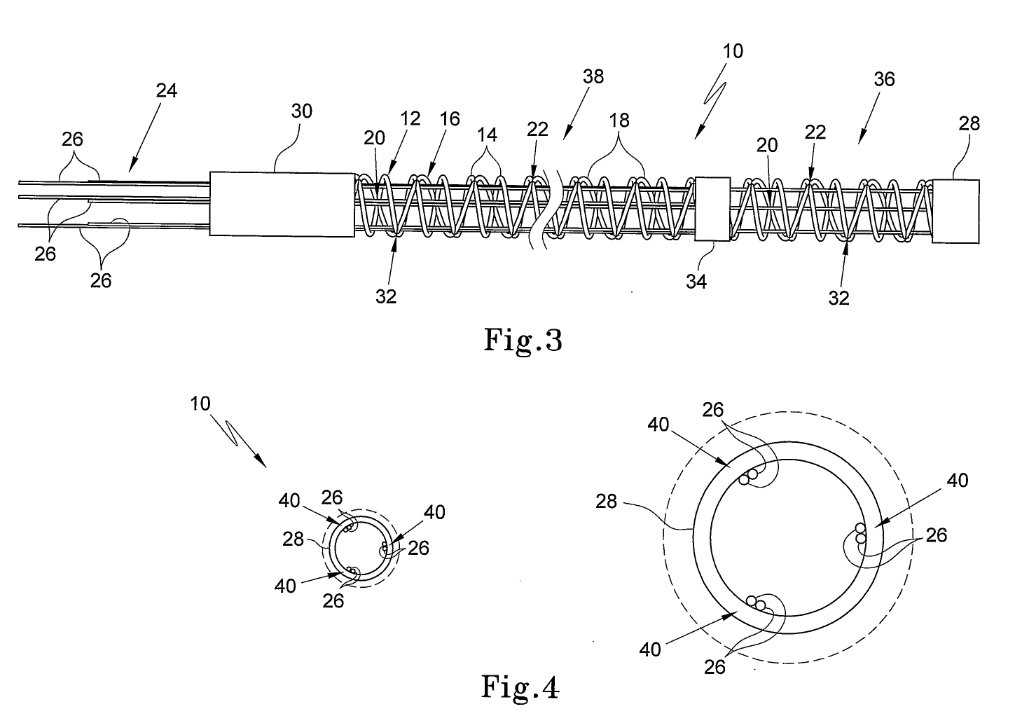

Referring now to FIGS. 2 to 4 of the drawings, a second embodiment of a sleeve steering and reinforcing device 10 is described. With reference to FIG. 1 of the drawings, like reference numerals refer to like parts, unless otherwise specified.

Once again, the device 10 comprises two co-axially arranged elongate, helical elements 12, 16 with oppositely directed turns 14 and 18, respectively. However, in this embodiment, the pitch of the turns 14 of the helical element 12 is half that of the pitch of the turns 18 of the helical element 16. As a result, the turns 14 and 18 intersect at three zones 20, 22 and 32. Once again, the zones 20 are arranged along a first imaginary line, the zones 22 are arranged along a second, parallel, imaginary line and the zones 32 are arranged along a third, parallel, imaginary line. These imaginary lines are spaced from each other by 120°.

In this embodiment, the control arrangement 24 comprises at least three control wires 26. By appropriate manipulation of any one or two of the control wires 26 omnidirectional steering of the device 10 can be achieved.

In the embodiment shown in FIGS. 2 to 4 of the drawings, an intermediate collar 34 is arranged between the distal collar 28 and the proximal collar 30 to divide the device 10 into two sections 36 and 38. The section 36 is defined between the distal collar 28 and the intermediate collar 34 and the section 38 is defined between the intermediate collar 34 and the proximal collar 30. Each section 36 and 38 has its own set of three control wires 26 so that, in effect, the control arrangement 24 comprises six control wires 26. As shown more clearly in FIG. 4 of the drawings, the control wires 26 are arranged in three groups 40 of two control wires each, the groups 40 being spaced from each other by 120° and coinciding with the three imaginary lines.

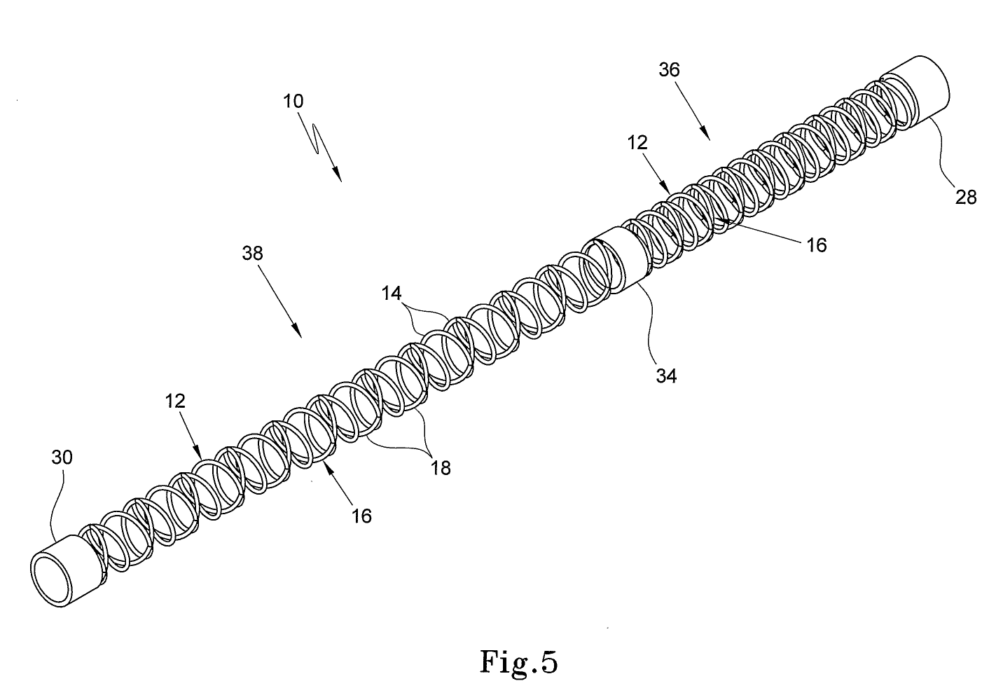

FIG. 5 of the drawings shows yet a further embodiment of the device 10. Once again, with reference to the previous drawings, like reference numerals refer to like parts unless otherwise specified.

In this embodiment, the section 36 has two helical elements of 12 and 16 where the helical elements 12 and 16 have pitches which differ from each other as described above with reference to FIGS. 2-4. Similarly, the helical elements 12, 16 of the section 38 differ in pitch from each other. However, the pitch of the helical element 12 of the section 36 differs from the pitch of the helical element 12 of the section 38. Similarly, the pitch of the helical element 16 of the section 36 differs from the pitch of the helical element 16 of the section 38. With this arrangement, different radii of curvature of bending of the sections 36 and 38 are obtained. It will further be appreciated that, rather than having the helical elements 12, 16 of constant pitch in each section 36, 38, the pitch of the helical elements 12, 16 could vary along the length of each section 36, 38 to provide a variable radius of curvature of bending of the sections 36, 38 of the device 10. It is also to be noted that the variable pitch helical elements 12, 16 can be applied in the FIG. 1 embodiment to provide a device 10 having a variable radius of curvature of bending along its length.

Further, the pitch of one of the helical elements 12 or 14 may be maintained constant while the pitch of the other helical element 12 or 14 may vary along the length of the device 10 or section 36, 38, as the case may be. The zones at which the turns of the helical elements intersect are then arranged along spirals to facilitate a twisting motion of the device 10.

As indicated above, with the provision of the two sections 36 and 38 of the device 10, the sections 36 and 38 can be steered independently of each other facilitating maneuvering of a sleeve, incorporating the device 10 through the vascular system of a patient to arrive at the site. Also, as indicated above, the provision of oppositely directed turns 14 and 18 of the helical elements 12, 16, respectively, of the device 10 facilitates transmission of torsion should it be necessary to do so. An additional benefit of the second embodiment of the invention is, however, that a clinician does not need to rotate a proximal end of the catheter assembly in order to facilitate in-plane bending. With the omnidirectional steering able to be achieved by the device 10 of the second 35 embodiment, it is not necessary for the clinician to rotate the catheter assembly to steer through the vascular system of the patient.

The device 10 is formed from a work piece of a suitable material. Typically, the work piece is a length of a tubular member from which material is removed, for example, by laser cutting to form the intersecting turns 14, 18 of the helical elements 12, 16, respectively, so that the turns 14 and 18 intersect. The collars 28, 30 and, where applicable, 34 can also be formed integrally with the helical elements 12, 16, as a one-piece unit, from the same length of tubular member. The material from which the device 10 is made is a suitable, resiliently flexible biocompatible material such as stainless steel, nitinol, titanium, or the like. A suitable synthetic plastics material, such as, for example, nylon, could also be used. It will be appreciated that, in this case, the elements 12 and 16 have the same diameter.

Instead, the device 10 could be fabricated by two nested helical elements 12 and 16. In the latter case, the inner element has an outer diameter closely approximating the inner diameter of the outer element to be a snug or interference fit in the outer element.

It is an advantage of the invention that a sleeve steering and reinforcing device 10 is provided which facilitates reinforcing of a sleeve while permitting steering of a distal end of the sleeve and which facilitates transmission of torsion. A further advantage is that the device 10 can be arranged in an electrode sheath of the catheter itself to facilitate steering of a distal end of the electrode sheath of the catheter. This is particularly advantageous when used in conjunction with the Applicant's method of manufacturing an electrode sheath of a catheter as described in the Applicant's International Patent Application No. PCT/AU01/01339 dated 19 Oct. 2001 and entitled “An electrical lead”. The contents of that International Application are incorporated in this specification by reference. As described in that specification, the lumen of the electrode sheath is unimpeded by electrode conductors so that the steering wires 26 can be arranged within the lumen while still providing a small diameter electrode sheath. Those skilled in the art will appreciate that the smaller the diameter of an elongate device such as the electrode sheath or an introducer carrying the electrode sheath, the easier it is to steer the elongate device through the vascular system of a patient.

Yet a further advantage of the invention is that, because the device 10 can be embedded in the sleeve itself, it is not necessary to include any further sleeves over a flexible end of the device to inhibit the ingress of foreign material into the interior of the sleeve. This therefore reduces the cost of a catheter assembly incorporating the device.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1: A sleeve steering and reinforcing device which includes:
a first, elongate, helical element having spaced turns; and
at least one further, elongate, helical element co-axially arranged with the first helical element, the at least one further helical element having a plurality of spaced turns wound in an opposite direction to the turns of the first helical element such that the turns of the elements coincide at predetermined zones, the zones being arranged along lines extending parallel to a longitudinal axis of the elements.

2: The device of claim 1 in which the elements are formed by working a tubular work piece of appropriate material.

3: The device of claim 2 in which the work piece is worked by removing material to define the turns of the helical elements.

4: The device of claim 2 in which the coinciding zones of the elements are zones of intersection of turns of the elements.

5: The device of claim 1 in which two helical spring-like structures are co-axially arranged to provide the coinciding zones.

6: The device of claim 1 in which the lines lie out of a plane of stiffness, the plane of stiffness passing through a longitudinal axis of the elements.

7: The device of claim 6 in which the pitch of the turns of one of the helical elements is the same as the pitch of the turns of the other helical element.

8: The device of claim 7 in which the coinciding zones of the turns are arranged along lines which are spaced 180° from each other, the lines lying in a plane of binding passing through the longitudinal axis of the elements and the plane of bending being orthogonally arranged relative to the plane of stiffness.

9: The device of claim 6 which comprises two helical elements of oppositely directed turns with the pitch of the turns of one of the helical elements being different from the pitch of the turns of the other helical element.

10: The device of claim 9 in which the pitch of the turns of one of the helical elements is twice the pitch of the turns of the other helical element.

11: The device of claim 10 in which three lines of coinciding zones of the turns are formed with adjacent lines being spaced 120° apart.

12: The device of claim 8 which includes a control arrangement for controlling bending of a distal end of the device.

13: The device of claim 12 in which the control arrangement comprises a plurality of elongate control members.

14: The device of claim 13 in which two control members are provided with the two members lying in the plane of bending.

15: The device of claim 9 which includes a control arrangement for controlling bending of a distal end of the device.

16: The device of claim 15 in which the control arrangement comprises a plurality of control members.

17: The device of claim 16 in which three control members are provided with adjacent control members being spaced 120° apart.

18: The device of claim 13 in which a stiffening collar is arranged at least at a distal end of the elements with a distal end of each control member being secured at or adjacent the collar.

19. The device of claim 18 in which at least one further collar is arranged proximally of the distal collar.

20: The device of claim 19 in which a set of control members is associated with the at least one further collar as well as with the distal collar to provide a plurality of independently controllable sections.

21: The device of claim 1 in which the helical elements are of metal.

22: The device of claim 1 in which the helical elements are of a polymeric material.

23: The device of claim 22 in which properties of the polymeric material of at least one of the helical elements differ along the length of the at least one helical element to vary characteristics of the at least one helical element along its length.

24: The device of claim 1 in which a pitch of the turns of at least one of the helical elements varies along the length of the at least one helical element to provide a variable radius of curvature of bending along the length of the device.

25: The device of claim 1 in which a pitch of the turns of at least one of the helical elements varies along the length of the at least one helical element and the zones at which the turns of the helical elements intersect are arranged along spirals to facilitate a twisting motion of the device.

26: A sleeve which includes:
an elongate tubular member; and
a sleeve steering and reinforcing device, as claimed in claim 1, carried by the tubular member.
